(19) United States
(12) Patent Application Publication
(10) Pub. No.: US 2010/0055044 A1
(43) Pub. Date: Mar. 4, 2010

(54) Ruthenium compounds and compositions

(76) Inventor: Binyomin A. Cohen, Brooklyn, NY (US)

Correspondence Address: DILWORTH & BARRESE, LLP, 1000 WOODBURY ROAD, SUITE 405, WOODBURY, NY, 11797, US

(21) Appl. No.: 12/322,884

(22) Filed: Feb. 9, 2009

Related U.S. Application Data
(60) Provisional application No. 61/063,977, filed on Feb. 8, 2008.

(51) Int. Cl. A61K 9/12; A61P 35/00; A61K 47/48

(52) U.S. Cl. 424/43; 424/641

(57) ABSTRACT

Ruthenium containing compounds and compositions which inhibit the cellular mitochondrial electron transfer mechanism by providing electrochemically-generated oxygen to an oxygen-deprived environment. Also provided are pharmaceutical compositions containing the same and methods for treating cancer using the pharmaceutical compositions.

### CROSS-REFERENCE TO RELATED APPLICATIONS

This application claims priority to U.S. Provisional Application Ser. No. 61/063,977 filed Feb. 8, 2008, which is herein incorporated by reference.

### FIELD OF THE INVENTION

The present invention relates to Ruthenium compounds and compositions and Technetium altered by neutron bombardment to produce artificial Ruthenium compounds and compositions, and more particularly to polymeric molecular adducts of diRuthenium/diRuthenium (diRu/diRu) which inhibit the cellular mitochondrial electron transfer mechanism during the series of mutagenesis events commonly associated with malignant cancers. Providing electrochemically generated oxygen to an oxygen-deprived environment can effectively treat a cancerous state, whether metastasis or fixed.

The present invention also relates to pharmaceutical compositions comprising the Ruthenium and Technetium altered to artificial Ruthenium compounds of the present invention as well as methods for preventing or treating cancer comprising the administration of a therapeutically effective amount of the pharmaceutical compositions of the present invention alone or in combination with other compositions for the treatment of cancer and or cancer related illnesses.

### BACKGROUND OF THE INVENTION

Cancer is not just one disease but, a large group of almost one hundred diseases. Its two main characteristics are uncontrolled growth of the cells in the human body and the ability of these cells to migrate from the original site and spread to distant sites. If the spread is not controlled, cancer can result in death.

One out of every four deaths in the United States is from cancer. It is second only to heart disease as a cause of death in the States. About 1.2 million Americans are diagnosed with cancer annually; more than 500,000 die of cancer annually. Cancer can attack anyone. Since the occurrence of cancer increases as individual's age, most of the cases are seen in adults, middle-aged or older. Sixty percent of all cancers are diagnosed in people who are older than 65 years of age. The most common cancers are skin cancer, lung cancer, colon cancer, breast cancer (in women), and prostate cancer (in men). In addition, cancer of the kidneys, ovaries, uterus, pancreas, bladder, rectum, and blood and lymph node cancer (leukemias and lymphomas) are also included among the 12 major cancers that most affect Americans.

Cancer, by definition, is a disease of the genes. A gene is a small part of DNA, which is the master molecule of the cell. Genes make “proteins,” which are the ultimate workhorses of the cells. It is these proteins that allow our bodies to carry out all the many processes that permit us to breathe, think, move, etc. Throughout people's lives, the cells in their bodies are growing, dividing, and replacing themselves. Many genes produce proteins that are involved in controlling the processes of cell growth and division. An alteration (mutation) to the DNA molecule can disrupt the genes and produce faulty proteins. This causes the cell to become abnormal and lose its restraints on growth. The abnormal cell begins to divide uncontrollably and eventually forms a new growth known as a “tumor” or neoplasm (medical term for cancer meaning “new growth”).

In a healthy individual, the immune system can recognize neoplastic cells and destroy them before they get a chance to divide. However, often-mutant cells escape immune detection and survive to become malignant tumors or cancers. Tumors are of three types, benign, precancerous and malignant. A benign tumor is not considered cancer. It is slow growing, does not spread or invade surrounding tissue, and once it is removed, it doesn't usually recur. A precancerous and or malignant tumor, on the other hand, is for all intent and purposes cancer. It invades surrounding tissue and spreads to other parts of the body. If the cancer cells have spread to the surrounding tissues, then, even after the malignant tumor is removed, it generally recurs.

Many drugs and therapies exist to treat cancer, however since so many different types of cancers exist, treating cancer presents many challenges. One challenge in treating cancer is to develop a drug and/or treatment that target cancerous cells while allowing non-cancerous cells to be largely unaffected. Another is to provide a potent drug that effectively kills the cancerous cells once targeted without poisoning the environment around the cancerous cells. Still yet another challenge is to provide a drug that is able to get into the intracellular environment of the cancerous cells in order to stop further proliferation of the cancerous cells and kill the existing cancerous cells.

Improving the delivery of drugs and other agents to target cells and tissues has been the focus of considerable research for many years. Though many attempts have been made to develop effective methods for importing biologically active molecules into cells, both in vivo and in vitro, none has proved to be entirely satisfactory. Optimizing the association of the drug with its intracellular target, while minimizing intercellular redistribution of the drug, e.g., to neighboring cells, is often difficult or inefficient.

Most agents currently administered to a patient parenterally are not targeted, resulting in systemic delivery of the agent to cells and tissues of the body where it is unnecessary, and often undesirable. This may result in adverse drug side effects, and often limits the dose of a drug (e.g., glucocorticoids and other anti-inflammatory drugs) that can be administered. By comparison, although oral administration of drugs is generally recognized as a convenient and economical method of administration, oral administration can result in either (a) uptake of the drug through the cellular and tissue barriers, e.g., blood/brain, epithelial, cell membrane, resulting in undesirable systemic distribution, or (b) temporary residence of the drug within the gastrointestinal tract. Accordingly, a major goal has been to develop methods for specifically targeting agents to cells and tissues. Benefits of such treatment include avoiding the general physiological effects of inappropriate delivery of such agents to other cells and tissues, such as uninfected cells resulting in toxic damage to non-infected cell and tissues.

Intracellular targeting may be achieved by methods and compositions that allow accumulation or retention of biologically active agents inside cells. Many of the current treatment regimes for cell proliferation diseases such as psoriasis and cancer utilize compounds that inhibit DNA synthesis. Such compounds are toxic to cells generally but their toxic effect on rapidly dividing cells such as tumor cells can be beneficial.

Three main mechanisms operate during a cancerous condition, which are abnormal to the cellular system and immune functioning. In one mechanism, the cellular mitochondria contain less oxygen than is needed, such that normal mitochondria have an impaired ability to transfer electrons as needed, resulting in the production of free radicals via an electron-rich environment in great numbers. These high-energy electrons cause an alteration in the mitochondrial membrane potential, with increased Calcium production and alteration in proton generation. In the second mechanism, the normal inhibition of telomere length by the ribonucleic enzyme telomerase is hyper-produced; telomerase allows the continued production of telomeres of incorrect length, which results in shortened chromosomes, as seen in multiple types of cancer cell lines. Additionally, oxygen is deprived in cancerous cells due to the hypermetabolic state the cell is driven into by the repetitive cell lines generated as daughter cells, even where oxygen is still present, due to the number of cancerous cells and/or a level of hypermetabolisis. In cancerous cells, cell nuclei are pycnotic, and cellular fibrin lines degenerate. Tumor cells have a higher rate of glycolysis under aerobic conditions than do most non-tumor cells. Moreover, many tumors have high rates of glucose use and lactic acid production in the presence of oxygen, due to a variety of mechanisms, including membrane transport differences and variations in ATP regulation with pH levels that are continually acidic.

Cancerous cells do not occur in a healthy, oxygenated environment. Indeed, cells in hypermetabolic states lack oxygen. This cellular hypoxia, while unknown on the gross anatomic, clinical level is the foundation for precancerous cells. The lack of cellular oxygen lack is the primary sequence that provides all cancers with a greater ability to mutate and generate further malignancies.

Accordingly, a variety of methods to introduce oxygen into the body have been developed, including pressure chambers, liquid oxygen, peroxide, chemical compounds, acid/alkaline balancing, injections, and ozone treatments. Indeed, flooding cells with oxygen may retard the growth of cancer cells or may even help them return to normal, however providing too much oxygen to the actual involved site, i.e., at cell, “in situ” or tissue region cause the production of superoxidants in normal tissue and destruction of lung and viable tissue parenchyma, as clearly displayed in Retrolential Fibroplasia (R.F.) of the newborns on concentrations of 40% oxygen, and less time, when breathed at above 40% for longer than six (6) hour periods.

As such providing oxygen via airways cannot provide oxygen at the level of precancerous or malignant cellular or tissue sites, as this method of administration will not allow enough Oxygen to fulfill and supplement the pre and cancerous cells hypermetabolic needs which cause to generate anaerobic metabolism in the tumor and adjacent to tumor cell's.

One embodiment of the present invention involves compounding a diRuthenium polymetallate attached to a diRuthenium polymetallate attached to a suitable carrier molecule. In another embodiment of the present invention, a diRuthenium sawhorse molecule is attached to a diRuthenium polymetallate attached to a suitable carrier to provide Oxygen at and on site via the proximity of the two Ruthenium atoms which generate Oxygen gas from a water system provided by the mammalian interstitial, and plasma fraction off of either blood and or tissue water. As such, the compounds of the present invention provide an oxygen-evolving-center on a cancerous site, as needed.

The main purpose of the present invention is to provide a tissue and/or cancerous hypemetabolic site a consistent renewable supply of fresh oxygen, without the loss of the Oxygen gradient normally encountered. The Ruthenium atoms simultaneously absorb excess high-energy electrons and alter the low oxygen ratio to high energy electrons which lead to reactive oxygen species (ROS) and further mutagenic species within such mitochondrial-electron-chain alterations.

Being hyperproduced due to the damaged proton gradients further impacting the AATP losses already encountered by the cancers pulling most of the available oxygen. The proton gradient is also the generated system that accounts for ATP production. Therefore, what is needed in the market today are compounds, pharmaceutical compositions and methods of treating cancer that provide healthy, normal levels of oxygen to targeted cells within an oxygen-deprived environment so as to disable primary and secondary bio-cascades which increase or generate greater mutagenicity of pre-cancerous and cancerous cells, thus preventing the proliferation of existing cancerous cells while at the same time maintaining a healthy level of an oxygenated environment for hypermetabolic precancerous and cancerous cells.

The present invention provides a new class of organometallic compounds using bonded diRuthenium atoms to inhibit the cellular mitochondrial electron transfer mechanism of electron rich species to compensate for the reduced oxygen levels normally found in hypermetabolic states surrounding pre-cancerous and cancerous tissues. The electron rich species together with low oxygen level results in reduced oxygen with greater electrons per molecule, which produce free radicals, and reactive oxygen species (ROS) via the electron chain transfer via mitochondrial subsystems, which increase mutagenicity but provide energy anerobically with reduction in ATP.

In pre-cancerous and cancerous cells however, energy is derived from oxide species that are free radical generating. Indeed, in oxygen deficient sites, pre-cancerous and cancerous cells or tissue derive energy from mutagentic forms, such as electron rich oxygen (ROS), for example, further increasing mutagenicity and simultaneously disabling the immune systems responsive subsystems.

If, however, electrochemically generated oxygen were provided to a cell in an oxygen-deprived environment, subsequent proliferation of a pre-cancerous or cancerous cell may well be prevented, and sequestration of the Ca++ signaling pathway averted. Therefore allowing the pre-cancerous or cancerous cells to their normal, non-mutated state to be directed under immunity and prior DNA sequencing to be secured to normal.

### SUMMARY OF THE INVENTION

The present invention relates in general to polymeric molecular adducts of Ruthenium comprising compounds and compositions and Technetium altered by neutron bombardment to produce artificial Ruthenium compounds that inhibit the cellular mitochondrial electron transfer mechanism by converting water from cellular and tissue plasma to electrochemically generate oxygen in an oxygen-deprived environment. In addition, the present invention relates to pharmaceutical compositions comprising at least one of the Ruthenium compound of the present invention as well as methods of treating a mammal, namely a human, using the same.

In particular, one object of the invention is to provide a composition comprising an adduct of a compound having the general formula [X]-L-[X] and at least one type POM represented herein by the formula Na₁₄[Ru₂Zn₂(H₂O)₂(ZnW₉O₃₄)₂] and or Na₁₄[Ru₂Zn₂(H₂O)₂(ZnW₉O₃₄)₂] then substituted to Ru₂Zn₂(H₂O)₂(ZnW₉O₃₄)₂⁻¹⁴ then substituted to an electrogenerating catalyst POM as seen below,

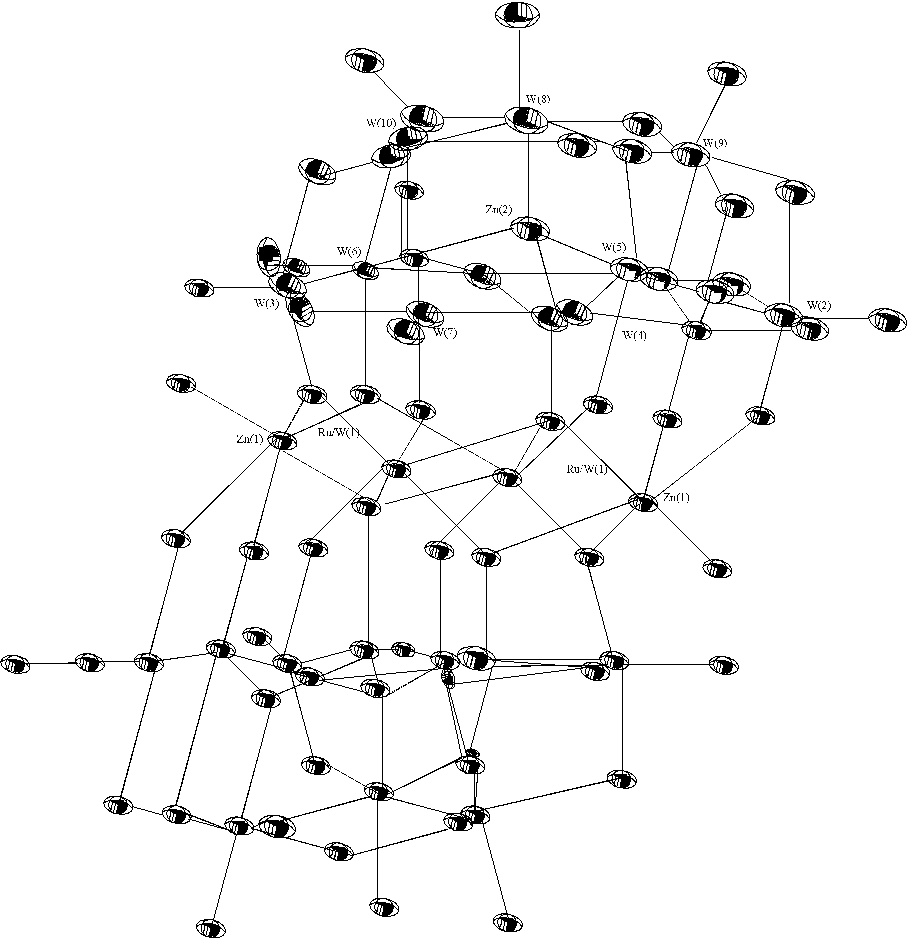

The POM is attached to at least one biological carrier wherein [X] is a diruthenium sawhorse represented by the formula (Ru₂(CO)₄(u-n²-O₂CR)₂)ⁿ wherein −3≦n≦6 and R represents hydrogen, hydroxide or a substituted or unsubstituted alkyl, heteroalkyl, aryl or heteroaryl group; and L is a linking group that bonds the 2 [X] complexes together.

Another object of the invention is to provide a composition for treating cancer comprising an effective amount of a composition comprising an adduct of a compound having the general formula [X]-L-[X] and at least one POM represented by the formula Na₁₄[Ru₂Zn₂(H₂O)₂(ZnW₉O₃₄)₂] and/or Na₁₄[Ru₂Zn₂(H₂O)₂(ZnW₉O₃₄)₂] then substituted to Ru₂Zn₂(H₂H)₂(ZnW₉O₃₄)₂^{-14,} attached to at least one biological carrier wherein [X] is a di-ruthenium sawhorse represented by the formula (Ru₂(CO)₄(u-n²-O₂CR)₂)ⁿ wherein −3≦n≦6 and R represents hydrogen, hydroxide or a substituted or unsubstituted alkyl, heteroalkyl, aryl or heteroaryl group; and L is a linking group that bonds the 2 [X] complexes together and one or more pharmaceutically acceptable carriers and/or adjuvants.

Still yet another object of the invention provides a method for treating cancer in a mammal comprising administering to a mammal in need of cancer treatment a therapeutically effective amount of the composition for treating cancer comprising an effective amount of a composition comprising an adduct of a compound having the general formula [X]-L-[X] and at least one POM represented by the formula Na₁₄[Ru₂Zn₂(H₂O)₂(ZnW₉O₃₄)₂] and or Na₁₄[Ru₂Zn₂(H₂O)₂(ZnW₉O₃₄)₂] then substituted to Ru₂Zn₂(H₂O)₂(ZnW₉O₃₄)₂^{-14,} attached to at least one biological carrier wherein [X] is a di-ruthenium sawhorse represented by the formula (Ru₂(CO)₄(u-n²-O₂CR)₂)ⁿ wherein −3≦n≦6 and R represents hydrogen, hydroxide or a substituted or unsubstituted alkyl, heteroalkyl, aryl or heteroaryl group; and L is a linking group that bonds the 2 [X] complexes together and one or more pharmaceutically acceptable carriers and/or adjuvants.

The compositions can be in the form of capsules, tablets and/ or solutions for oral administration; parenterally in the form of sterile solutions or suspensions, in some cases intravenously in the form of sterile solutions, or suspensions, and topically in the form of solutions, suspensions or ointments, and by aerosol spray, as well as with/without a propellant for nasal administration.

The present invention is described in greater detail in the following Detailed Description section and in the claims.

### DETAILED DESCRIPTION OF THE INVENTION

The present invention provides a new class of organometallic compounds comprising bonded Ruthenium atoms, which can be used to inhibit the cellular mitochondrial electron transfer mechanism, by providing electrochemically-generated oxygen to an oxygen-deprived environment.

In effect cancer and precancerous cells and or tissue have signaling mechanisms that are activated by one such pathway during hyper metabolic cells, the oxygen is sucked into the cancerous and or pre-cancerous cells at greater rates than normal cells with two clear and obvious sequela, first the cellular region is oxygen starved, second the pre-cancerous cell pushes the high energy electrons off their oxygen carriers. For example, either off hemoglobin or another carrier dissolved in the blood plasma, which results in a pathway of increased cytoplasm Ca⁺⁺. This also leads to damaged Mitochondrial membrane potential, diminished ATP production via loss of proton gradient concentration, less oxygen and hydrogen ion bound up as end product of the electron chain transfer system of the Mitochondria as the end product water. All leading to telomerase enzymes being signaled on, this to protect the rapidly increasing daughter cells is generated from the cancerous cells and or tissue.

A key finding being that telomerase appears to be “the” mechanism that maintains telomere length and enabling cell division to continue indefinitely in cancer cells. In Science (Vol. 266, P. 2011-2014, Dec. 23, 1994), scientists found that telomerase was active in 98 out of 100 immortal tumor cell lines and in 90 out of 101 malignant tumor specimens, representing twelve (12) tumor types. As such factually Mitochondria are by virtue of such electron transfer mechanisms the primary source of free radicals.

The mitochondrial electron transport chain is achieved by four large metalloproteins bound to the inner membrane of the mitochondria, namely Complexes I through IV. Each complex, except Complex II, pumps protons across the membrane. Electron transfer between complexes is achieved via the mobile coenzymes ubiquinone from Complexes I through III within the lipid membrane. Cytochrome C is derived from Complex III and IV. However In a preferred embodiment of the present invention, the DiRuthenium molecular adducts function as either one of two specific D shell molecular elemental atoms. In particular, the preferred molecular adducts are a multiple diRuthenium electro-generated oxygen catalyst.

Cytochrome A, cytochrome B, and cytochrome C are compounds that undergo rapid oxidation, (loss of electrons) and reduction, (gain of electrons). Cytochrome A receives electrons transferred from ubiquinone where cytochrome A carries the electron to cytochrome oxidase complex, because Oxygen has high affinity for electrons. As such, the lower amounts of oxygen found in cancerous hypermetabolic states allow for a proportionally higher amount of electrons per oxygen than in a healthy state. Thus the available oxygens are able to obtain additional electrons. Normally the release of a large amount of free energy, as when oxygen is reduced to water, allows for the abstraction of the hydrogen as a proton transferred in the electron transfer chain from the NADH as NAD+H⁺ and 2 electrons. These electrons are passed on to oxygen, which is then reduced to water. If this does not occur, there is no sink for protons to pass along their electrons which will remain bound to NADH and effectively block the transfer of electrons, which together with the electrons carried by cytochrome A, allow for the build up of electrons on the compounds and oxygen species in the area which absorb such increases resulting in ROS. The blocking of cytochrome A and its carrying electrons involves a porphyrin ring containing an iron (Fe) center.

Cytochrome A, cytochrome B and cytochrome C are of a family of colored proteins all related by the presence of a bound heme group. Hemoglobin carries the bulk of oxygen in the body and allows for the reduction to carry the Hydrogen ion from tissues as Co² is dissolved in cellular water. As such, cytochrome A allows for oxygen carrying on the heme portion of the protein-globin, which alters the pH of the local environment. The molecules of the present invention are configured to be in close proximity to adjust and correct the cytochrome A overload of electrons pumped into the cell and tissue from cancers excessive absorbing almost all the oxygen available, leaving little for cytochrome A to allow proton generation.

Once diatomic oxygen (O₂) picks up an electron it is highly reactive and seeks more electrons (typically thought four e⁻), which allow it to become even more reactive, as a radical species. A cell can use oxygen only because cytochrome oxidase holds onto oxygen at a special bimetallic center where it remains locked up between a heme-linked iron (Fe) and a copper atom until it has picked up a total of four electrons. Only then can the two oxygen atoms (O₂) of the oxygen molecule be safely released as two molecules of water. It is this water that allows the proton (H⁺) gradients that produce most of the cells ATP.

Ruthenium is the D-shell orbit electron absorber and allows for the transfer of electrons, which allow the D-shell and lower energy state orbital pre-D-shell orbital, to first fill the lower ‘px’, ‘py’ and ‘pz’ subshells within the ‘P orbital’, prior to filling the D-shells, which act like electron sponges without effecting the oxidation state of the two Ruthenium atoms. As the D-shell orbit slowly fills, maintenance of the atomic orbits are maintained via the D-shell which absorbs excess electrons, as is found in ROS and other electro-donating species. Thus, the D-shells of the diRuthenium compounds of the present invention also protect against the hyper Ca²⁺ state pumped into the cytoplasm by cancers, which affect the cellular organelles via a signaling mechanism that leads to apoptosis of cells and tissue. The excessive electron-rich generated species allow for prolonged telomerase enzyme activity to preserve the rapid splitting daughter cell lines generated by malignant cancers. It is the ratio of p53 and telomerase that halts the cells rapid division, but allows the telomerase enzyme to maintain and continue.

Accordingly, some cancers take decades to grow and alter to malignant species from precancerous tumors having almost zero activity, due to the halting mechanism of the p53 on a smaller amount of telomerase enzyme. Indeed the ratio between p53 and telomerase is initially about 1.0:0.6 to about 1.0:0.8, however the ratio between p53 and telomerase reverses as the p53 is consumed and declines in blood level reaching an inverted ratio of about 0.8:1.0 to about 0.8:1.6. This results in malignancies heretofore unseen. As such, it is this specific usage of Ruthenium atoms as dimers and a diatomic modality species i.e., a diRuthenium:polyoxometallate to bind the excess free calcium (Ca⁺⁺) due to its semi-chelating effect.

The metal centers and other cofactors of the metalloproteins bound to the inner membrane of the mitochondria carry electrons from the reducing products of the citric acid cycle (NADH, succinate) to dioxygen. Accordingly, sufficient oxygen must be available to oxidize the NADH, which functions as a Lewis base.

Due to the inevitable leakage of electrons from the mitochondria in the electron transport chain, mitochondria are a source of free radicals. Since free radicals are highly reactive, they may interact with DNA and proteins to alter cellular functions.

Other chemical carcinogens do exist and can be typically classified into two groups: genotoxic and non-genotoxic carcinogens. While genotoxic chemical carcinogens interact with the DNA of the host, non-genotoxic chemical carcinogens do not. Chemical carcinogenesis is a multi-step construct. Indeed, while genotoxic chemical carcinogens are unreactive by themselves, and hence do not directly cause cancer, genotoxic chemical carcinogens are often procarcinogenic and proximate carcinogens, which are converted to electron deficient intermediates, including primary or ultimate carcinogens. In particular, to convert genotoxic chemical carcinogens to primary or ultimate carcinogens, the cytochrome P450-dependant monoxygenases found on the endoplasmic reticulum (ER) alters the proximate carcinogens to reactive electron-deficient intermediates (electrophiles). These reactive intermediates next undergo an interaction between electron rich (nucleophilic) regions I and at the DNA to produce a mutation. Such interaction between the between electron rich (nucleophilic) regions I and the DNA is an initial step in chemical carcinogenesis. The DNA may revert to normal if DNA repair mechanisms can operate successfully. If not, the transformed DNA within the cell will affect the mitochondria to produce electron-rich free radicals, which cause the cell to grow into a cancerous tumor.

As demonstrated above, the ability of genotoxic chemical carcinogens to convert to primary or ultimate carcinogens is dependent upon the ability of electron-rich regions to access electron-deficient regions. Indeed, in primary or ultimate carcinogens, the electron transport mechanism within the mitochondrion is turned off, which prevents electron-rich regions to access electron-deficient regions.

Two mechanisms operate during a cancerous condition, both of which are abnormal to the cellular system. In one mechanism, the cellular mitochondria contain less oxygen than is needed, such that normal mitochondria have an impaired ability to transfer electrons, resulting in the production of free radicals via an electron-rich environment in great numbers. In the second mechanism, the normal inhibition of telomere length by its ribonucleic enzyme telomerase allows the continued production of telomeres of incorrect length wherever oxygen is deprived.

It is well known that tumor cells have a higher rate of glycolysis under aerobic conditions than do most non-tumor cells. Moreover, some tumors have high rates of glucose use and lactic acid production in the presence of oxygen, which may be due to a variety of mechanisms, including membrane transport differences and variations in ATP regulation.

It has been demonstrated that cancerous cells do not occur in a healthy, oxygenated environment. Indeed, it has been proposed that lack of oxygen is the primary cause of all cancers. Accordingly, a variety of methods to introduce oxygen into the body have been developed, including pressure chambers, liquid oxygen, peroxide, chemical compounds, acid/alkaline balancing, injections, and ozone treatments. Indeed, flooding cells with oxygen may retard the growth of cancer cells or may even help them return to normal.

The mitochondrial ribosomes of cancerous cells often contain less oxygen than is needed because they have an altered structure, which functions to diminish the ability of the cell to produce oxygen, therefore accounting for their limited aerobic potential. In that tumor cells possess insufficient catalase and peroxidase, they are incapable of effective peroxide inactivation, therefore resulting in peroxide intolerance. However, when tumor cells are exposed to ozone, a significant decrease in lactate content is found, indicating therefore that ozone induces metabolic inhibition in carcinomas.

Indeed, in one study, cultured cells of different carcinoma types were compared with non-cancerous human lung fibroblasts exposed to ozonated air (0.3, 0.5, and 0.8 parts per million (ppm) of O³ for 8 days). Sweet, et al., Ozone Selectivity Inhibits Cell Growth of Human Cancer Cells, Science (1980) 209:931-933. Alveolar adenocarcinoma, breast adenocarcinoma, uterine carcinosarcoma, and endometrial carcinoma showed 40% cell growth inhibition at 0.3 ppm and 60% cell growth inhibition at 0.5 ppm. In cancerous cells exposed to 0.8 ppm, 90% cell growth inhibition was found. These results demonstrate that cancer cells have a decreased ability to compensate for the oxidative challenge of ozone as compared to non-cancerous cells.

As discussed above, due to the inevitable leakage of electrons from the electron transport chain, mitochondria are a source of free radicals. Since free radicals are highly reactive, they may interact with DNA and proteins to alter cellular functions. Indeed, free radicals and the shortening of telomeres both contribute to the aging process and/or cell death. Although new cells may regenerate by cell division, the telomere will become shorter in the daughter cells absent a sufficient quantity of telomerase to extend the telomere.

Telomerase is an enzyme critical in maintaining the structural integrity, positioning, and accuracy in the replication of chromosomes. Telomerase, which maintains telomere length, also enables cell division to continue indefinitely in cancer cells. As the length of the telomere is decreased, it will activate p53 to stop the cell division if a sufficient amount of telomerase is available. In an oxygen-deprived environment however, telomerase is signaled to be hyper-produced, even if the telomeres being produced are shorter in length than their parent cells. Accordingly, cancerous cells, which involve an oxygen-deprived environment, will cause telomerase to be hyper-produced, resulting in the proliferation of new cells with telomeres that are shorter in length than their parent cells. This results in cell destruction sequences and apoptosis, however telomerase allows such shortened cells to be sustained.

The excessive oxygen consumption by cancerous cells is a natural defense mechanism, used to provide hypermetabolic cancerous cells in producing rapid daughter cells. And utilizing the Oxygen for its own needs rather than the immune defenses the TNF and Ca2+ signaling for immune assistance provides. Additionally due to the heat generated by the production of new cells oxygen's loss of electrons allow the cancer to implement the electrons into its own energy supply even and especially so when the oxygen levels fall as they must in malignant pre and cancerous states. Accordingly, cancerous cells utilize indirect oxidation whereby the electrons are passed through a series of metal clusters and cofactors, including flavins, iron-sulphur clusters, cytochromes, and copper centers. These groups are embedding in large membrane-bound proteins, and the passage of electrons through them is coupled to the transport of protons across the membrane, from the interior matrix space of the mitochondria to the inter-membrane space. The energy released by the electron flow allows the recombination of the oxygen down the mitochondrial-electron-chain to bind with the proton (H⁺), which creates a proton concentration gradient across the mitochondrial membrane sustaining a healthy potential. The potential energy inherent in the proton concentration gradient is used to synthesize ATP when protons flows back across the membrane through another enzyme complex, ATP synthase.

Local increases in oxygen at cancerous sites disturb and damages cancerous tissue growth. Indeed, as discussed in greater detail herein below, providing oxygen to an oxygen-deprived cancerous cell prevents telomerase from being hyper-produced, therefore allowing p53 to be activated, which stops cell division. Indeed, the cancerous cell will cease mutagenesis and die as a result of tumor necrosis factors released by the immune system.

Cancerous cells demand more oxygen than is available, due to the hyper-metabolic consumption of oxygen by the cancerous cell(s). Thus, the cancerous cell(s) will be starved of oxygen, causing the mitochondria of the cellular cancer and its nucleic mitochondria to increase its mutagenicity. As mutagenesis increases, electron dumping increases as well, as does lactic acid, and free radicals, all via anaerobic metabolism. If the cancerous cells and the adjacent cellular matrix platforms are deprived of oxygen for long enough, free radicals will be released, which increases the amount of telomerase delivered to the cell(s) allowing further cancerous growth, despite the lack of oxygen.

The ability to oxidize by adding and supplementing the lost oxygen from the local environment involves the reduction of cytochrome P450 reductase within the mitochondrial walls, along with the transfer of such reducing power by oxidation involving the oxidized species loss of electrons to the cytochrome. Accordingly, the ability to destroy cancerous cells is directly related to the level of available oxygen being at a cellular site.

By introducing either compounds or compositions to generate oxygen electrochemically in an oxygen-deprived cancerous cell, oxygen is released to a cellular matrix platform and its sustaining neoplastic growth, as the need for oxygen arises, thus preventing telomerase from being recruited to the cell, therefore minimizing the cancerous growth. Healthy cells adjacent to the matrix platform will not sense any oxygen diminution.

Conversely the usage of an oxygen-evolving-center via electro-catalytic conversion of cellular water to oxygen is also considered this as preventing against ROS generated free radicals. This as Di-Ruthenium compounds and compositions of the present invention control oxygen-production, such that reactive oxygen species are not paradoxically generated via the Oxyhemoglobin Dissociation Curve (ODC) in the body, and the Oxygen generation on site. In particular, the ODC fixates p50 to within narrow ranges, specifically 23 torr/atm to 27 torr/atm at the pH ranges compatible with life in the blood, and blood brain barrier. As the pH becomes more acidic (lower pH from pH 7.35 down to 7.24, 7.15, 7.05 ) the p50 curve shifts and the oxyhemoglobin curve releases more oxygen at less p50, i.e., from 23 torr/atm-27 torr/atm→15 torr/atm to 20 torr/atm.

Ruthenium is one known organometallic oxygen generator. As discussed above, organometallic oxygen generators are useful to treat cancerous cells in that providing oxygen to an oxygen-deprived environment (such as that of a cancerous cell), which prevents telomerase from being hyper-produced therefore allowing p53 to be activated, which stops cell division. A single Ruthenium in a tetrachloride ring has been developed and used for cancer treatment. (See, Groessi, et al., Structure-Activity Rrelationships for NAMI-A-type Complexes (HL)[trans-RuCIL(S-dmso)ruthenate(III)] L=Imidazole, 1,2,4-Triazole, 4-Amino-1,2,4-triazole, and 1-Methyl-1,2,4-triazole): Aquation, Redox Properties, Protein Binding, and Antiproliferative Activity, J. Med. Chem., (2007) 50(9):2185-2193.) However, these compounds containing a single Ruthenium are locked in Ca⁺² via the four Cl⁻ atoms, and therefore prevent these compounds from generating oxygen electrochemically to meet the needs of a cell. In addition, Ruthenium is known to sequester excess calcium generated in hypercalcemic states. The additional calcium found in conditions of inflammatory disease and in tumorgenesis signals cellular and tissue apoptosis due to a destabilization in mitochondrial membrane potential, which directly effects membrane permeability causing the cell to send catalase to trigger apoptosis.

The destabilization in mitochondrial membrane potential also prevents oxidative phosphorylation within the mitochondrial membrane from occurring. In that oxidative phosphorylation within the mitochondrial membrane generates the majority of cellular adenosine triphosphate (ATP), failing to provide a mechanism to sequester excess calcium generated in hypercalcemic states results in diminished ATP production. Indeed, the low levels of oxygen prevent the production of ATP, which is manufactured by the mitochondria via the electron chain Cp450 pathway. In particular, oxygen is rerouted to generate free electrons, which may be used by a cancerous cell. The free electrons in the region produce reactive oxygen species, which lead to mutagenesis, and hence cancer.

Moreover, compounds containing a single Ruthenium fail to absorb free radicals so as to prevent super-oxygenation, which is known to cause cancerous growth. Accordingly, because compounds containing a single Ruthenium in a tetrachloride/Ca⁺² arrangement are inadequate to inhibit the cellular mitochondrial electron transfer mechanism by failing to provide electrochemically generated oxygen to an oxygen-deprived environment, and further fail to absorb free radicals, they fail to provide effective treatment to cancerous cells.

However, as discussed in greater detail hereinbelow, it has been discovered that molecular adducts of Ruthenium containing compounds, preferably diRuthenium/diRuthenium, provide electrochemically generated oxygen to an oxygen-deprived environment, therefore making such Ruthenium complexes effective in the treatment of cancerous cells.

In one embodiment, the compound of the present invention is a Ruthenium complex bound to a carrier. Indeed, in order to get the Ruthenium complex into the intracellular matrix of the targeted cancerous cell, the Ruthenium complex of the present invention must be attached to a biologically acceptable carrier molecule. A biological acceptable carrier is a carrier that is able to transport the Ruthenium complex across biological barriers (including lipophilic and hydrophilic barriers) by attaching long chain hydrocarbons to the carrier along with the porphyrin to deliver the Ruthenium complex of the present invention to a target site, such as, for example, a neoplastic target site. Many different mechanisms can be used to accomplish this task, including reactor cell binding, passive transport, active transport, multi-drug resistance, and phagocytosis. Biologically acceptable carriers include but are not limited to porphyrin, hemoglobin, heme-containing complexes, casein, and synthetically made carrier molecules. The preferred biological carrier is porphyrin.

In a preferred embodiment of the present invention, porphyrin is used as the biologically acceptable carrier molecule for a variety of reasons including the fact that porphyrins are naturally occurring, and have the desired ability to cross biological barriers (including lipophilic and hydrophilic barriers) and deliver compounds across biological barriers to a target site, such as, for example, a neoplastic target site even with long hydrocarbon side chains attached.

Porphyrins are naturally occurring heterocyclic macromolecules characterized by the presence of one pyrroline and three pyrole chemical groups interconnected via their a carbon atoms via methine bridges (═CH—). Porphyrins are aromatic and possess 4n+2 pi electrons delocalized over the macromolecule. The simplest porhpyrin, porphine, is also the parent compound.

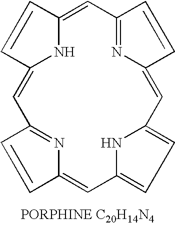

Porphyrins bind metals to form complexes. The metal ion, which usually has a charge of 2+ or 3+, is the central N₄ cavity formed by the loss of two protons. Porphyrin-based compounds have the ability to cross biological barriers (including lipophilic and hydrophilic barriers), and hence have the ability to deliver compounds across biological barriers to a target site, such as, for example, a neoplastic target site.

Different methods can be used to synthesize a Ruthenium into a porphyrin. One method is to use a soluble transition metal catalysts which include phosphane and carbon monoxide ligands. Phosphane ligands offer increased catalyst stability, improved reaction rates and selectivities, and enhanced partitioning into two-phase systems.

As a preparation, Ruthenium atoms ranging from Ru5+ to Ru3+, are placed anaerobically under an inert atmosphere, such as, for example, Argon gas. The Nitrogen, found biologically within the porphyrin ring, is inserted into the inert atmosphere, previously dissolved in polar solvent of a diRuthenium sawhorse molecule having a carbonyl compound. The carbonyl compound is used as a ligand to bridge the oxygen atoms where the double-bond oxygen atoms are, and is then released at several positions (sites 21 and 23) into the porphyrin ring. In particular, the CO π orbitals lie only slightly above the porphyrin (πi) orbital. Additionally, the Ru—C bond is shortned, and the CO(π) orbitals are brought in closer contact with the porphyrin ring, thus compensating for the bond length to provide an oxygen-evolving center.

The use of prior oxygenated porphyrin compounds is not excluded in the preparation described above prior to Ruthenium replacement of the rings Nitrogen atoms.

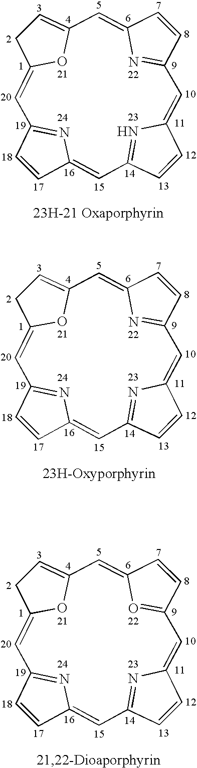

The oxygen atoms bonded to sites 21 and/or 22 (as classified by IUPAC) define the “legs” of the diRuthenium sawhorse molecule. DiRuthenium atoms arranged in a sawhorse structure was first described by G. R. Cooks, B. F. G. Johnson, J. Lewis 1969, Inorg. Phys. Theor., Part VII.

Under NMR spectroscopy (220 Mc./sec) the Ruthenium complexes show signals which are assigned to coordinated carboxylate groups. The structure of the Ruthenium complexes are defined as follow:

M₃(CO)₁₂→[M(CO)₂(RCO₂)]n→M₂(CO)₄(RCO₂)₂L₂

(wherein (M=Ru) and R=H, Me, Et, or n-C₉H₁₉)
The two carboxylate ligands serve as bridges between the metal atoms, and the six carbonyl groups occupy terminal positions. All reactions were carried out under purified nitrogen, using analytical grade reagents.

In the diRuthenium sawhorse molecule, it was found two molecules of O₂CR serve as the bridging ligands, and the polymers and dimers react with ligands such as tertiary phosphines, triphenylarsine or pyridine to produce dimeric complexes of the type M₂(CO)₄(RCO₂)₂L₂, where the acetate complex reacts with carbon monoxide under mild conditions to produce a dimeric complex Ru₂(CO)₆(MeCO₂)₂.

As a novel application the diRuthenium sawhorse molecule of the present invention may be formed using the cold plasma brush technique pioneered by Yixiang Duan, C-CSE, C. Huang and Q. S. Yu.: Department of Chemical Engineering, Center for Surface Science and Plasma Technology, University of Missouri-Columbia, Columbia, Mo. 65211 MS K484, Los Alamos National Laboratory, Los Alamos, N.M. 87545 in which a cold plasma brush is generated at atmospheric pressure with low power consumption in the level of several watts (as low as 4 W) up to tens of watts (up to 45W). The plasma can be ignited and sustained in both continuous and pulsed modes with different plasma gases such as argon or helium, but argon was selected as a primary gas for use in this work. The brush-shaped plasma is formed and extended outside of the discharge chamber with typical dimension of 10-15 mm in width and less than 1.0 mm in thickness, which are adjustable by changing the discharge chamber design and operating conditions.

The brush-shaped plasma provides some unique features and distinct nonequilibrium plasma characteristics. Temperature measurements using a thermocouple thermometer demonstrate that the gas phase temperatures of the plasma brush are close to room temperature (as low as 42° C.) when running with a relatively high gas flow rate of about 3500 ml/min. For an argon plasma brush, the operating voltage is from less than 500 V to about 2500 V, and the argon gas flow rate may be varied from less than 1000 to 3500 ml/min. The cold plasma brush can most efficiently use the discharge power as well as the plasma gas for material and surface treatment. The very low power consumption of such an atmospheric argon plasma brush provides many unique advantages in practical applications including battery-powered operation and use in large-scale applications.

As used in the present invention, the brush-shaped plasma formed extends outside of the discharge chamber with typical dimensions of 10-15 mm in width, and less than 1.0 mm in thickness, which are adjustable by changing the discharge chamber design and operating conditions. This argon plasma brush is used to denude the porphyrin of its Nitrogen atoms and to utilize a carbine ligand to bind two Ruthenium atoms of the sawhorse diruthenium molecule binding of the two Ru³⁺ atoms, affecting a neutral charge to the molecule. This also allows for lipolicity of the molecule keeping its polar nature bound thus allowing tumors of predominantly fat to be accessible to the compound.

Notably, the temperature measurements using a thermocouple thermometer should be for the gas phase temperatures of the plasma brush at close to room temperature, preferably as low as 42° C., so as not to discharge the carbine molecule being bound by the carbine sawhorse diRuthenium polyoxomettalate group linked to the porphyrin molecule.

As shown below two O₂CR ligands bridge the Ruthenium atoms and hold the Ruthenium atoms in place. In particular, the two Nitrogen atoms of the porphyrin are deprotonated and hence carry a negative charge, which creates a strong bond between the two anionic nitrogens and the Ruthenium atoms, thus ensuring that the Ruthenium atoms remain bound to the porphyrin ring. This is essential since free Ruthenium can cause severe health problems and even death of the patient. Thus, the oxygen atoms on the legs of the diRuthenium sawhorse molecule replace the hydrogen of the N—H.

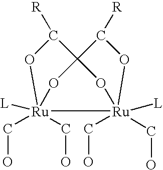

The first use of Scanning Probe Microscopy (SPM) was the Scanning Tunneling Microscope (STM) developed in the late 1970s and early 1980s by Gerd Karl Binnig and Heinrich Rohrer at an IBM research lab in Zurich, Switzerland, earning these scientists, along with Ernst Ruska, the 1986 Nobel in Physics. The STM was initially used as an imaging device, capable of resolving individual atoms by recording the quantum tunneling current that occurs when an extremely sharp conductive probe tip (usually tungsten, nickel, gold, or Pt) is brought to within about one atomic diameter of an atom, and then adjusting the position of the tip to maintain a constant current as the tip is scanned over a bumpy atomic surface.

A height change as small as 0.1 nm can cause tunneling current to double. The tip is connected to an arm that is moved in three dimensions by stiff ceramic piezoelectric transducers that provide sub-nanometer positional control. If the tip is atomically sharp, then the tunneling current is effectively confined to a region within ˜0.1 nm of the point on the surface directly beneath the tip, thus the record of tip adjustments generates an atomic-scale topographic map of the surface. STM tips can scan samples at ˜KHz frequencies, although slower scans are used for very rough surfaces. In some modern STMs (e.g., the DI Nanoscope), the sample is moved while the tip is held stationary.

Wong and colleagues initially had prepared nanotube tips by oxidation in air at 700° C., burning off all but 2% of the original material and leaving the ends covered with carboxyl (COOH) groups whose chemistry is rich and well understood. Four different kinds of tips were created: (1) the original carboxyl tip, which is acidic; (2) an amine-terminated tip (made by forming an amide bond to one of the amine groups in ethylenediamine (H₂NCH₂CH₂NH₂)), which is basic; (3) a hydrocarbon-terminated tip (made by forming an amide bond to benzylamine (C₆H₅CH₂NH₂)), which is hydrophobic.

Wong's tips had three closely related advantages over previous techniques. First, when a functional group is attached to the apex of an Si₃N₄ or SiO₂ tip, these groups usually adhere to the sides of the tip as well; upon using the tip as a tool, there is a constant hazard that contact with the sides of the tip will alter the work piece in unwanted places. Unlike the tips built with bulk techniques, the ends of Wong's tips are very different from their sides—the carboxyl groups are attached only on the ends of the nanotube, not on the sides. Second, Wong's tips have lateral dimensions set by the nanoscale dimensions of nanotubes, not by top-down fabrication techniques. The small effective radius of nanotube tips significantly improves resolution beyond what can be achieved using commercial silicon tips. Lateral resolution of <3 nm can be achieved by using COOH-terminated single-walled nanotubes tips. Third, single-walled nanotube tips are much closer to yielding truly single atom tips with controlled chemistry than any other alternative. A (10, 10) nanotube with a 1.4-nm diameter has just 20 atoms at an open end. Given a statistical distribution of tubes with varying numbers of carboxyl groups attached to their ends, one can build a ligand which covers the whole end of the tube, therefore providing a method for ensuring that just one molecule of known structure and orientation was present at the end of the probe.

The third general pathway leading to molecular manufacturing involves a technology known as scanning probe microscopes (SPMs). A major limitation of the STM was that it only worked with conducting materials such as metals or semiconductors, but not with insulators or biological structures such as DNA. To remedy this situation, in 1986 Binnig, Quate and Gerber developed the Atomic Force Microscope (AFM) which is sensitive directly to the forces between the tip and the sample, rather than a tunneling current. An AFM can operate in at least three modes. In “attractive” or non-contact mode (NC-AFM, 0.01-1 N/m force constant), the tip is held some tens of nanometers above the sample surface where it experiences the attractive combination of van der Waals, electrostatic, or magnetostatic forces. In “repulsive” or contact mode (CAFM, 0.01-1 N/m force constant), the tip is pressed close enough to the surface for tip and sample electron clouds to overlap, generating a repulsive electrostatic force of ˜10 nN, much like the stylus riding a groove in a record player. There is also intermittent-contact mode (IC-AFM, 0.01-1 N/m force constant), which is sometimes called “tapping” mode. In any of these modes, a topographic map of the surface is generated by recording the up-and-down motions of the cantilever arm as the tip is scanned. These motions may be measured either by the deflection of a light spot reflected from a mirrored surface on the cantilever or by tiny changes in voltage generated by piezoelectric transducers attached to the moving cantilever arm. Typical AFM cantilevers have lengths of 100-400 microns, widths of 20-50 microns, and thicknesses between 0.4 to several microns. AFM tips may be positioned with ˜0.01 nm precision, compressive loads as small as 1-10 pN are routinely measured, and the tips may be operated even in liquids.

A SPM tip can ionize and extract atoms at site, i.e., within or on a carrier, such as Porphyrin, for example. A STM tip composed of two sides, for extraction of atoms via ionizing the atom chosen for removal and one side of the two sided SPM/STM tip for insertion by lowering the ionization current voltage across its surface. This allows for either the insertion of an atom, molecule or ion via data calculations for the particular species (atom, molecule, ion) which is desired to be inserted. One particular SPM/STM tip for preparing the compounds of the present invention has two sides separate from one another. Both sides of the tip, (tip sides), have a diameter of about 0.9 nm, 0.45 nm per side insulated from the other by Silicon Nitride and/or diamond atoms having a thickness of about 2 nm, which creates a barrier to each side of said tip.

STM technology has also improved, reaching resolutions of ˜0.001 nm in the z direction (vertical) and ˜0.01 nm in the xy plane, well beyond atomic resolution. The STM remains the instrument with the best resolution. By 1998, the growing family of SPMs included at least forty types of instruments and techniques that relied on interactions between a scanned surface and a nearby probe. Different instruments measured different forces and thus could be used to characterize different properties of the surface. For example, friction force microscopes (FFMs), magnetic force microscopes (MFMs), shear force microscopes (ShFMs), scanning capacitance microscopes (SCMs), scanning conducting ion microscopes, chemical force microscopes, and electrostatic force microscopes (EFMs) measured frictional drag or other binding forces. Magnetic resonance force microscopes (MRFM) used a field generated from a small magnet mounted on the tip of the cantilever arm to probe nuclear magnetic moments in a small region on the surface of the sample, imaging atom types and even detecting the spin of a single electron. By the mid-1990s, AFMs were already a $100 million/year industry and SPMs generally were an off-the-shelf technology costing up to $50,000-$500,000 for complete systems, with the whole industry worth up to ˜$0.5 billion annually.

To manufacture atomically precise parts, it also will be necessary to manipulate covalent bonds at the probe tip. STMs have broken and created chemical bonds; ˜1 volt pulses were used to pull atoms out of crystals, binding them to the tip, and then to reinsert them back into the crystal. In 1995, the first demonstration of catalysis on a nanometer scale was reported by scientists at the Molecular Design Institute at LBL. They used an AFM modified to function like an ultrafine-point pen for catalytic calligraphy to change the chemical composition of a material surface one molecule at a time. A surface was prepared as a self-assembled monolayer (SAM) of alkalized molecules capped with a crown of three nitrogen atoms, then platinum-coated chromium was deposited onto an AFM silicon tip just a few atoms wide. The SAM was soaked with a hydrogen-containing solvent, then scanned by the AFM over a 100 micron² area, with the platinum catalyzing a covalent bonding reaction in which hydrogen was added to the azides, transforming them into amines as revealed by selective fluorescent tags.

“IBM Almaden” has experimented with removing individual atoms from metal surfaces using an STM hooked into a Virtual Reality Data glove apparatus. The calculations for application of the compounds and compositions of the present invention to modal anticancer where regio-selectivity is a major obstacle for chemical synthesis has been determined by the following methodology. Where atom or molecule “A” requires 799 KJ/mole for bond energy, either dissociation or association, then the division value of 6.02×10²³ is divided into the 799 Kilojoules=X, where X is the approximate single joule or multiple of joule value to access the bond energy either for extraction or insertion. For extraction of an atom or a molecule “A,” X is equal to the value minus 0.002 to 0.006 of that current, due to the electrostatic charge build up across said tip. For insertion of an atom or a molecule “A,” X is that joule value divided by 0.002 to 0.006 due of that current, due to charge attraction created when the tip is removed from the insertion site, which protects the molecule or atom inserted. Accordingly, two methods are proposed to insert ruthenium within a carrier, such as the four available nitrogen sites of a porphyrin ring, for example.

The present invention involves a dimer of diRuthenium attached to a histidine molecule which can be utilized as an anticancer drug, such as seen below. The starting material may be the Sodium Phthalocyanine, from “Strem chemicals,” where a diRuthenium polyoxometallate is inserted into the nitrogen atom sites a Porphyrin ring, replacing the same. It is understood that the diRuthenium polyoxometallate may be inserted into Nitrogen sites which are either “cis” or “trans.” When using phthalocyanine or porphyrin rings, as a starting material, for example, the metal complex is to the pyrrole (inner rings) and not the peripheral nitrogen. The side arm on the peripheral carbons is the benzyl amide of the amino-acid histidine (which can be R or S). R is the mirror image of S.

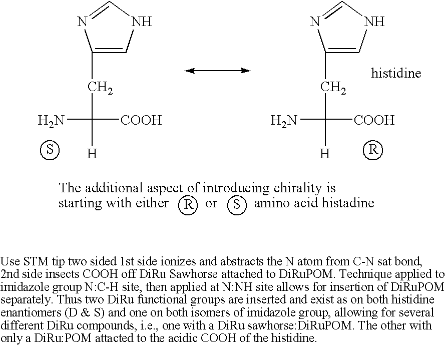

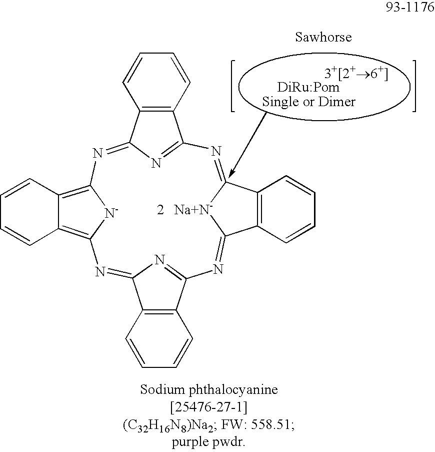

In one embodiment of the present invention, argon ions are used to strip the Nitrogen atoms. However, it is understood that other types of atoms may be used. In a preferred embodiment of the present invention, the heterocyclic porphyrin ring is exposed to an argon ion plasma stream to denude the nitrogen atoms within the porphyrin ring for ruthenium-oxygen insertion. Methods for denuding the nitrogen atoms to insert ruthenium-oxygen have been described by Yixiang Duan., C-CSE, C. Huang and Q. S. Yu.: Department of Chemical Engineering, Center for Surface Science and Plasma Technology, University of Missouri-Columbia, Columbia, Mo. 65211 MS K484, Los Alamos National Laboratory, Los Alamos, N.M. 87545. This procedure can be used to produce the Ruthenium containing compounds of the present invention.

The synthesis of porphyrin starts with freshly distilled pyrrole and an aldehyde. The latter can be p-amino benzaldehyde. Preferrably, p-cyanobenzaldehyde is used. The synthesis of porphyrin is accomplished according to the following process: (a) protecting the aldehyde; (b) reducing the cyano to aminomethyl; (c) protecting the amine; (d) deprotecting the aldehyde; (e) treating the resulting aldehyde with the pyrrole in the presence of Zinc Chloride, resulting in a zinc coordinated porphyrin; (f) deprotecting the amine; (g) treating the resulting compound with an amine protected amino-acid such as histidine (to add an imidazole group which would mimic hemoglobin) using standard peptide synthesis methods; (h) removing the zinc; and (i) treating the resulting porphyrin with Ruthenium. The resulting compound will have the imidazole group fill one of the coordination centers of Ruthenium. If the Ruthenium is in the bivalent state, the complex is neutral and will not interact with the polymetallate. If the Ruthenium is trivalent however, then the complex has one positive charge and can form a compound with the polymetallate.

Alternatively, Ruthenium dimers with porphyrin may be prepared according to the following reaction mechanism:

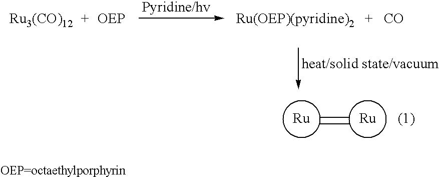

The molecular structure of the dimer (1) in the above reaction mechanism has a Ru—Ru bond distance of about 2.408 Angstroms.

Oxidative addition of RₙEERₙ to the Ruthenium dimer produced by the above reaction mechanism yield M-Erₙ bridged dimers as follows:

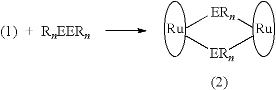

where: E=P, S, Se, or Te; n=1, or 2; R=alkyl, or aryl.

Reactions of (1) with chalcogens produce μ₂-E bridged diRuthenium complexes (E=S, Se, Te). For example, in a Te-bridged complex, Te=P(i-Propyl)₃ may be used to introduce the Te bridge due to the very weak P—Te bond which results in a very reactive atomic tellurium when reacted in solution with appropriate metal complexes.

Notably, the separation of the two Ruthenium porphyrins may be attenuated by the choice of chalcogen, as demonstrated by the following reaction:

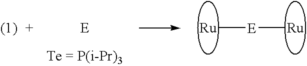

where E=S, Se, or Te. It has been found that a similar reaction with white phosphorus will yield the related μ-P dimer.

Alternatively, the -ERₙ bridged Ruthenium dimer may be formed first and the porphyrin introduced in a subsequent reaction using, for example, heat, light or the CO extrusion reagent, Me₃NO, to facilitate the substitution of the carbonyl ligands by the porphyrin, as demonstrated by the following reaction:

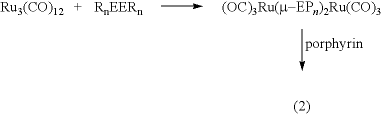

where: E=P, S, Se, or Te; n=1, or 2; R=alkyl, or aryl.

Once produced, the Ruthenium comprising compounds of the present invention can be used to inhibit the cellular mitochondrial electron transfer mechanism by providing electrochemically-generated oxygen to an oxygen-deprived environment in cancerous cells. The new class of organometallic compounds comprising bonded diRuthenium atoms of the present invention inhibits the cellular mitochondrial electron transfer mechanism by providing electrochemically-generated oxygen to an oxygen-deprived environment.

In one embodiment of the present invention, the present invention involves heretofore unknown polymeric molecular adducts of diRu/diRu. The diRu/diRu polymeric molecular adducts of the present invention interact with mitochondrial mechanisms and generate oxygen electrochemically. In particular, the increased oxidation electro-generated by the molecular adducts of the present invention function as oxygen-generating catalytic molecules. As discussed hereinabove, porphyrin-based compounds when attaching a long hydrocarbon side chain with the diRuthenium have the ability to cross biological barriers (including lipophilic and hydrophilic barriers), and hence have the ability to deliver compounds across biological barriers to a target site, such as, for example, a neoplastic target site.

Accordingly, one embodiment of the present invention involves molecular adducts of diRu/diRu covalently bonded within a porphyrin ring, the porphyrin ring being the biological carrier which delivers compounds across biological barriers to a target site. However, it is understood that other biological carriers capable of delivering diRu/diRu compounds across biological barriers to a target site may be used in accordance with the present disclosure, without departing from the spirit of the present invention.

The present invention involves diRuthenium sawhorse molecules bonded to a porphyrin and diRuthenium polyoxometallates bonded to porphyrin, which have surprisingly been found to provide electrochemically generated oxygen to an oxygen-deprived environment, therefore making diRuthenium sawhorse molecules and diRuthenium polyoxometallates effective in the treatment of cancerous cells. Indeed, diRuthenium sawhorse molecules and diRuthenium polyoxometallates attach a —SiR₂H functionality to the alkyl or aryl group on the bridging ligand in the generic dimeric structure:

Ru₂—SiR₂H+S_{urface} (of the polyoxometallate)_{→}Ru₂SiR₂O—Ru₂polyoxometallate+H₂.

Accordingly, the above reaction provides a synthetic process to covalently attach diRuthenium compounds to a diRuthenium polyoxometallate. For example:

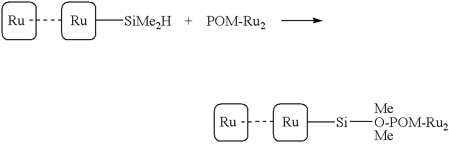

As demonstrated in the reaction above, the —SiMe₂H subsistent is contained on the porphyrin ligand, for use as a coupling group in a reaction with a surface oxide ligand on the polyoxometallate-diRuthenium complex to form a very stable Si—O bond to link the two diRuthenium molecules. However, it is understood that any alkyl or aryl may be included on the porphyrin ring, in place of Me₂, and used as the coupling group.

Moreover, the two diRuthenium molecules may be linked by other methods known in the art which provide bonding with an oxide surface including, for example, the formation of Si—O bonds, Sn—O bonds, or B—O bonds. The two diRuthenium molecules may also be linked by hydrogen bonding between a carboxylic or sulfonic acid functionalized diRuthenium porphyrin and the oxide surface of the polyoxometallate-diRuthenium complex, as demonstrated below:

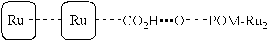

One embodiment of the present invention involves two monomers of a diRuthenium sawhorse (Ru₂(CO)₄(u-n²-O₂CR)₂)ⁿ. In one embodiment, up to four diRuthenium sawhorse molecules bonded to a porphyrin are utilized in the compounds of the present invention. In a preferred embodiment, two diRuthenium sawhorse molecules bonded to a porphyrin are utilized. Specifically, one nitrogen atom in the diRuthenium compound is on the same side as (cis), examples of sites within the porphyrin ring are sites 21 and 23 (as classified by IUPAC-see numbered formulas below). The other nitrogen atom in the diRuthenium compound is on the opposite side of (trans) as examples sites 21 and 23 (as classified by IUPAC—see numbered formulas below).

The DiRuthenium molecular adducts of the present invention function as either one of two specific D shell molecular elemental atoms. In particular, the preferred molecular adducts are a multiple diRuthenium electro-generated oxygen catalyst. Preferably, the D shell multiple atoms are bound by a ligand having a length of less than about 3.0 Angstroms. A ligand having a length of less than about 3.0 Angstroms will stress and tend to vibrate its intermolecular linkage and will, by such vibration, effectively interfere with a required cellular growth redox mechanism. For example some ligands that can be used in the present invention include tertiary phosphines, triphenylarsine or pyridine, i-Propyl, carbonyl ligands to bind to the porphyrin, sulfonic acid or carboxylic acid group to the porphyrin, or a surface oxide ligand on the diRuthenium polyoxometallate complex, porphyrin ligand (N+), (SO₄)²—, Cl—, F—, CH₃COO-(acetate), Phosphate PO₄³⁻, pyrazole groups (C₃H₄N₂).

In one embodiment of the polymeric molecular adducts of the present invention, a diRu molecule is joined with another diRu molecule to form a sawhorse molecule which is joined by short ligands to a molecule of diRu joined to another diRu molecule within a polyoxometalate (POM) group to form a “D shell” atom. In a preferred embodiment, the diRu/diRu sawhorse molecule is joined by ligands below about 0.275 Angstroms to an outer diRu/diRu molecule within a POM group.

In one embodiment, the present invention involves a diRuthenium polyoxometallate of the following formula:

RuZn₂,(H₂O)₂(ZnW₉O₃₄)₂]¹⁴

and usage of Na₁₄[Ru₂Zn₂(H₂O)₂(ZnW₉O₃₄)₂] substituted to Ru₂Zn₂(H₂O)₂(ZnW₉O₃₄)₂⁻¹⁴ as an electro-catalyst POM (which is disclosed in U.S. Pat. No. 7,208,244 herein incorporated by reference), which is bound to a biologically acceptable carrier. The diRuthenium polyoxometallate may be either a single molecule, or dimer.

In another embodiment, the present invention involves a porphyrin diRuthenium dimer having the formula 4Ru(III) Mesoporphyrin IX chloride (13-Bis(ethyl)-3,7,12,17-tetramethyl-21H,23H- porphine-2,18-dipropionic acid Tetraruthnate(III)chloride Mesohemin 8.

In yet another embodiment of the present invention the compounds of the present invention involve a diRuthenium sawhorse molecule bonded to a diRuthenium polyoxometallate, which is linked to a porphyrin ring or inhaled powder. In particular, the diRuthenium sawhorse molecule is the core of the diRuthenium complex and is covalently bonded across the 2(CO)₄ of the diRuthenium complex to the bridging Zinc atoms of the polyoxometallate.

In order to link the diRuthenium sawhorse molecule to the diRuthenium polyoxometallate on the diRuthenium-porphyrin complex, a bifunctional ligand is incorporated on the diRuthenium-porphyrin complex such that, the free Lewis base site on the ligand can bind, via a coordinate bond or hydrogen bonding, the surface oxide of the diRuthenium polyoxometallate to a sulfonic acid or carboxylic acid group on the diRuthenium-porphyrin complex. An example of this type of linking chemistry is illustrated below in scheme 1, which uses the favored binding of nitrogen ligands to Ruthenium and the favored oxygen binding of the oxophilic Sn(IV) center.

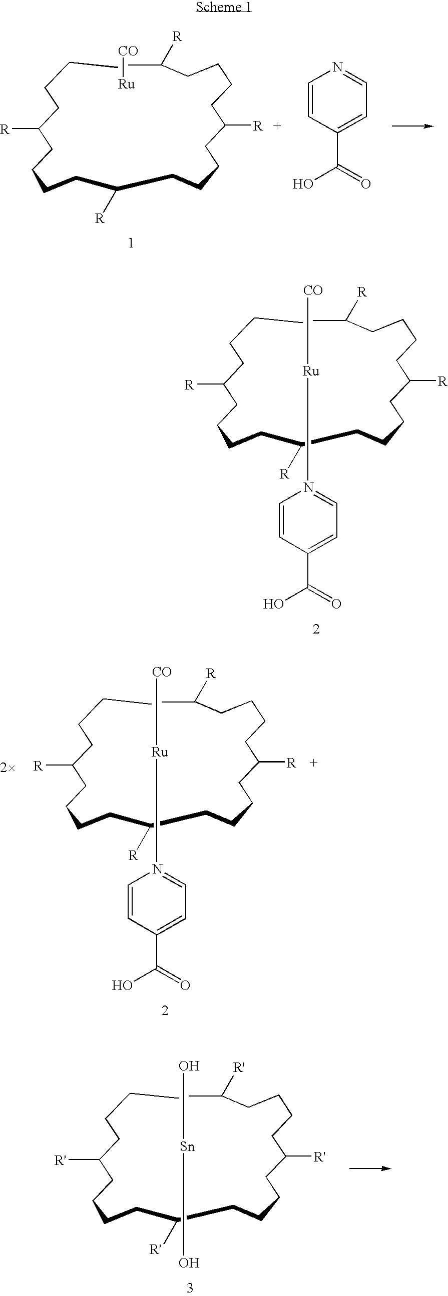

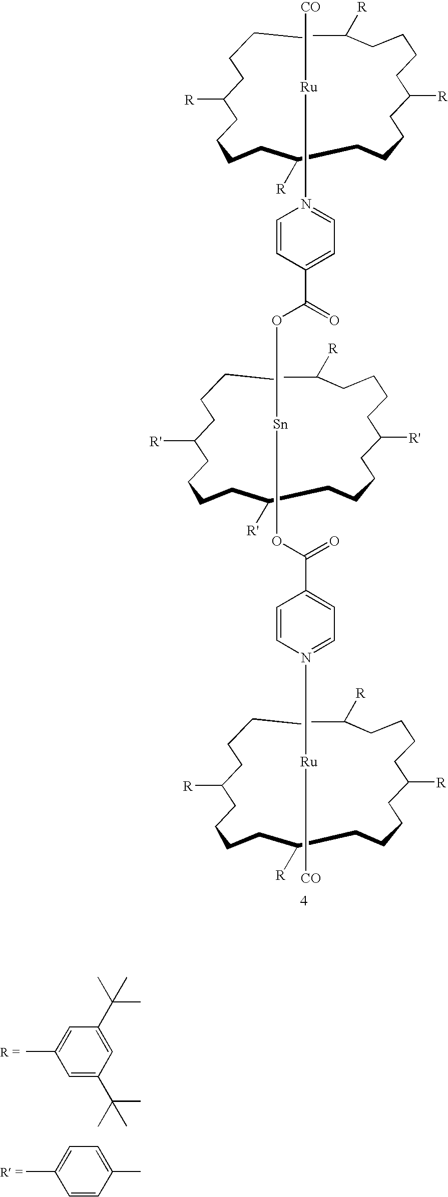

Alternatively, the diRuthenium sawhorse molecule may be covalently bonded to the diRuthenium polyoxometallate on the diRuthenium-porphyrin complex by taking advantage of the oxophilicity of silicon. In particular, a —SiR₂H functionality is attached to the alkyl or aryl group on the bridging ligand as demonstrated below:

Ru₂—SiR₂H+O_{surface} (of the POM)_{→}RU₂—SiR₂O—Ru₂POM+H₂

Alternatively, the —SiR₂H functionality could be put on the porphyrin ligand, or another auxiliary organic ligand, such as, for example, COOH—, CH3-, and 4OH—, which is incorporated in the diRuthenium dimer, for this reaction with surface oxide ligands in the diRuthenium polyoxometallate to form the very stable —Si—O— bond as a means of covalently linking the two diRuthenium complexes.

The diRuthenium sawhorse molecule may also be linked to the diRuthenium polyoxometallate on the diRuthenium-porphyrin complex by incorporating a bifunctional ligand on the diRuthenium-porphyrin complex such that the free Lewis base site on the ligand can bind the diRuthenium polyoxometallate via a coordinate bond or by hydrogen bonding of the surface oxide to a sulfonic acid or carboxylic acid group on the diRuthenium-porphyrin complex.

In another embodiment of the present invention, the diRuthenium sawhorse molecule or the diRuthenium polyoxometallate are presented as a homodimer, i.e, as a macrocycle or heterocyclic, wherein C is C2-C46, preferably C2-18, and C 36-46. In this embodiment, four Oxygen “legs” of the diRuthenium sawhorse molecule are bonded to a dimer as 2OC₃₄H₃₆ClRuN₄O₄:2OC₃₄H₃₆ClRuN₄O₄, to bond the Ruthenium complex to a biological carrier.

As discussed above, the ability of genotoxic chemical carcinogens to convert to primary or ultimate carcinogens is dependent upon the ability of electron-rich regions to access oxygen deficient regions generating increasing free radical and specifically ROS. Indeed, in primary or ultimate carcinogens, the electron transport mechanism within the mitochondrion is turned off, which prevents electron-rich regions access to electron-deficient regions.

Free radicals and the shortening of telomeres both contribute to the aging process and/or cell death. Although new cells may regenerate by cell division, the telomere will become shorter in the daughter cells absent a sufficient quantity of telomerase to extend the telomere. As the length of the telomere is decreased, it will activate p53 to stop the cell division if a sufficient amount of telomerase is available. In an oxygen-deprived environment however, telomerase is signaled to be hyper-produced, even if the telomeres being produced are shorter in length than their parent cells. Accordingly, cancerous cells, which generate and cause an oxygen-deprived environment, will cause telomerase to be hyper-produced, resulting in the proliferation of new cells with telomeres which are shorter in length than their parent cells, but are lengthened by the telomerase present therein.

Accordingly, the compounds and compositions of the present invention including diRu/diRu polymeric molecular adducts and mixtures thereof which utilize D orbital shells to bond to metal and/or oxometalate sites interfere with the electron gradient of the mitochondria to generate oxygen which prevents telomerase from being hyper-produced. This therefore allows the p53 to be activated, which stops cell division. Indeed, the cancerous cell will cease mutagenesis and die as a result of tumor necrosis factors released by the immune system. It is envisioned that the compounds and compositions of the present invention will only generate oxygen electrochemically when electrons are being transferred, and the energy usage is demanded from a highly radical cell, either during splitting and/or external cellular invasion.

For example, in a cancerous mass upon a cellular matrix platform in need of oxygen, the cellular matrix platform initially remains unchanged in that oxygen is in ample supply. However, when the cancer enters a hyper-metabolic phase, the cell begins to consume larger amounts of oxygen than normal. Accordingly, even if oxygen is available, the cancerous cell will cease mutagenesis and die as a result of the tumor necrosis factors released by the immune system.

In cancerous cells, the cells will demand more oxygen than is available, due to the hyper-metabolic consumption of oxygen by the cancerous cell(s). Thus, the cancerous cell(s) will be starved of oxygen, causing the mitochondria of the cellular cancer and its nucleic mitochondria to increase its Ca²⁺ levels damaging the mitochondrial membrane potential, signaling the dumping of SA1008 and SA1009 proteins and reactive oxygen species increasing further mutagenicity. As mutagenesis increases, electron dumping increases as well, as does lactic acid, and decreasing pH levels, increasing free radicals all via anaerobic metabolism. If the cancerous cells and the adjacent cellular matrix platforms are deprived of oxygen for long enough, free radicals will be released, which increases the amount of telomerase delivered to the cell(s) allowing further cancerous growth, despite the lack of oxygen.

By introducing either the compounds or compositions for treating cancer comprising diRu/diRu polymeric molecular adducts and mixtures thereof, and a pharmaceutically acceptable carrier of the present invention, oxygen is not only released electro-catalytically in ample supplies to a cellular matrix platform and its sustaining neoplastic growth on site, but the diRuthenium atoms as drug delivered tend to bind up the increased Ca²⁺ preventing signaling of deleterious cellular events previously mentioned. As the need for oxygen arises, and is met by the Ruthenium atoms generating oxygen on site, thus preventing telomerase from being recruited to the cell(s), therefore, minimizing the cancerous growth. Healthy cells adjacent to the matrix platform will not sense any oxygen diminution.

The level of oxygenation is determined by obtaining a sample of blood taken from the mammal in need of cancer treatment and running the blood sample through a Clark electrode at tissue temperature and recording the amount of oxygen therein. There is no need to provide oxygen to such oxygen-rich site, accordingly introducing either the compounds or the compositions of the present invention, will flood the area with a resulting electrophilic draw. Such a draw will prevent further growth of the cancerous cell(s). In particular, by attaching to the cancerous cell the compounds or the compositions of the present invention, the cancerous cell(s) will eventually suffocate due to the loss of electrons, which will prevent further growth and kill the cancerous cell(s). Indeed, the close multiple D shell atoms “lock up” the oxygen from the oxygen-rich environment, thus disabling the local cellular mitochondrial cytochrome electron transfer mechanism and the ability of the compound to bind and release oxygen.

The pH dictates the level of oxygen unload oxygen to the cell tissue. In other words, the pH dictates the flat end portion of the “Oxyhemoglobin dissociation curve” where PO₂ (that pressure needed to force or have the Oxygen carried upon the hemoglobin (Hgb) molecule, saturated at about 1.36 grams oxygen per gm Hgb. The same is true when Hgb is released to the tissue cells. In particular the pH dictates what pressure is necessary to reduced with Hydrogen, which occurs when CO₂ dissociates into Hydrogen ions. The Ph is the [−log] of the Hydrogen ion concentration used as a positive number. Loading and unloading oxygen is regulated by the local environment of the blood and tissue the Hgb is in contact with at each PO₂ level. P₅₀ is the level for the Oxyhemoglobin dissociation curve that is 50 percent saturated with Oxygen shifts in direct response to the pH encountered by the Hgb molecule. In cancers, especially malignant and proliferative pre-cancers the pH is commonly more acidic (lower pH) than normal.

The Ruthenium compounds and compositions of the present invention control oxygen-production, such that reactive oxygen species are not paradoxically generated via the oxyhemoglobin dissociation curve (ODC) and the Oxygen generation on site. In particular, the ODC fixates p50 at narrow ranges of 23 torr/atm to 27 torr/atm at the pH ranges compatible with life in the blood, and blood brain barrier. As the pH becomes more acidic (lower pH from pH 7.35 down to 7.24, 7.15, 7.05) the p50 curve shifts and the oxyhemoglobin curve releases more oxygen at less p50, i.e., from 23 torr/atm-27 torr/atm→15 torr/atm to 20 torr/atm.

The inability of pre-cancerous or cancerous cells to generate oxygen drastically reduces the available oxygen at such sites. Moreover, the additional calcium found in conditions of inflammatory disease and in tumorgenesis signals cellular and tissue apoptosis due to a destabilization in mitochondrial membrane potential, which directly affects membrane permeability causing the cell to send catalase to trigger apoptosis.

The present invention is also directed to a method of treating cancer comprising administering an effective amount of a pharmaceutical composition of the present invention comprising at least one Ruthenium comprising polymeric molecular adduct of the present invention, and/or mixtures thereof in a pharmaceutically acceptable carrier to a mammal in need of cancer treatment. In a preferred embodiment the mammal is a human. It has been discovered that an effective amount of Ruthenium comprising polymeric molecular adduct of the present invention and/or mixtures thereof in a pharmaceutically acceptable carrier are effective for the treatment of blood and solid tumors. Daily dosages provided to the mammal in need of treatment can be provided in a variety of different formats depending on the specific cancer being treated, the condition of the mammal being treated and the stage of the cancer. For example, the pharmaceutical composition of the present invention can be delivered to the mammal receiving treatment in the form of capsules, tablets and/or solutions for oral administration; parenterally in the form of sterile solutions or suspensions, in some cases intravenously in the form of sterile solutions, or suspensions, and topically in the form of solutions, suspensions or ointments, and by aerosol spray, as well as with/without a propellant for nasal administration.

As stated above, daily dosages provided to a mammal in need of treatment vary according to whether the Ruthenium compound of the present invention used in the pharmaceutical composition is a single Ruthenium atom, a Ruthenium dimer, etc. and the format being used to delivery the dosage. For dimeric Ruthenium anticancer drug dosage of 0.90 to 1.1 mg/pound of B.Weight (1.98 mg/kg to 2.42 mg/kg B.Weight ) for 11-16 days for two consecutive months dosage schedule of 4 days on 3 days off. For single DiRuthenium Porphyrin (carrier) drug as 1.26 to 2.05 mg/pound B.W.(2.77 mg/kg to 4.51 mg/kg B.Weight) per kilogram of patient per day for 11-16 days., 4 days on 3 days off. With plasma blood levels and urine excretion of Ruthenium fraction analyzed every third-day of treatment, this during clinical trials, which are mandatory by this inventor.

In another embodiment, the present invention involves an inhalable composition comprising administering an effective amount of diRu/diRu polymeric molecular adducts and mixtures thereof, and a pharmaceutically acceptable propellant. Indeed, it has been discovered that an effective amount of diRu/diRu polymeric molecular adducts and mixtures thereof, and a pharmaceutically acceptable propellant are effective for the treatment of ethmodial plate, sphinoidal, and retrosphinoidal tumors.

In one embodiment of the inhalable composition of the present invention, the pharmaceutically acceptable propellant is a gas. In another embodiment of the inhalable composition of the present invention, the pharmaceutically acceptable propellant is an aerosol. In still another embodiment of the inhalable composition of the present invention, the pharmaceutically acceptable propellant is a solvent. In a preferred embodiment of the inhalable composition of the present invention, the propellant is a saline having an additional Na+ ion charge-bonded to the Ruthenium compound, for use as a powder.

As discussed above, one embodiment of the present invention involves a heretofore unknown diRuthenium drug effective to treat cancer, which is utilized as an inhaled powder. In one embodiment, the DiRuthenium polyoxometallate complex is ionically bound to Na⁺ (Na=6-34) within an aerosol.

As stated above, daily dosages provided to a mammal in need of treatment vary according to whether the Ruthenium compound of the present invention used in the pharmaceutical composition is a single Ruthenium atom, a Ruthenium dimer, etc. and the format being used to delivery the dosage. For the inhalable powder diRuthenium drug of the present invention, a 0.03 ug/3 ml dose/day for 2 weeks has been determined effective to treat cancer in a mammal in need of treatment.

The inhalable powder diRuthenium drug of the present invention is prepared by crushing and sterilizing yellow crystals via airflow methodology. In particular, Di-u-acetato-hexacarbonyldiruthenium-catena-Di-u-acetatodicarbonylruthenium (1-15 g) linked to a diRuthenium polyoxometallate complex, where the Di-u-acetato-hexacarbonyldiruthenium-catena-Di-u-cetatodicarbonylruthenium is first suspended in n-hexane (5 ml) in a glass-lined autoclave. The suspended compound is then treated with carbon monoxide at 65-75 atm. and 65° F. with stirring for 4 hours, which yields pale yellow crystals suspended in a clear solution. Typically the yield would be quantitative, but, because of rapid decomposition through loss of carbon monoxide, recrystallization by typical methods is not possible. As such recystallization is carried out under increased barometric pressure (about 100 atm, for example), and at higher temperatures (about 150° F., for example).

Furthermore, for the deposition of Ruthenium on TiN films by metal-organic chemical vapor deposition, pretreatment of the TiN film surface with Electron Cyclotron Resonance (ECR) plasma is essential to enhance Ru nucleation. The effects of hydrogen, argon and oxygen plasma treatments on Ru nucleation, induced by Metal-Organic Chemical Vapor Deposition (MOCVD), were investigated using scanning electron microscopy, Auger electron emission spectroscopy and X-ray diffraction analyses. Ru nucleation is enhanced with increasing hydrogen or argon ECR plasma exposure time, while it decreases with increasing oxygen ECR plasma exposure time. Hydrogen ions in the hydrogen plasma react with TiN to form Ti and NH₃ on TiN when the underlying TiN surface is pretreated with argon ECR plasma.

Whole blood is made to pass over the active substrate where denitogenation occurs this via the “Argon ions” in the argon plasma remove nitrogen or oxygen atoms from the top surface of the TiN or TiON film during the argon plasma treatment. The highest Ru nucleation density was obtained on TiN when the underlying TiN surface is pretreated with argon ECR plasma.

While the above description contains many specifics, these specifics should not be construed as limitations of the invention, but merely as exemplifications of preferred embodiments thereof. Those skilled in the art will envision many other embodiments of varying types of DiRuthenium sawhorse molecules and of differing type POMs capable of electrogenerating Oxygen within the scope and spirit of the invention as defined by the claims appended hereto.

What is claimed is:

1. A composition comprising an adduct of a compound having the general formula [X]-L-[X] and at least one polyoxometallate (POM) represented by the formula Na₁₄[Ru₂Zn₂(H₂O)₂(ZnW₉O₃₄)₂] and/or Na₁₄[Ru₂Zn₂(H₂O)₂(ZnW₉O₃₄)₂] then substituted to Ru₂Zn₂(H₂O)₂(ZnW₉O₃₄)₂⁻¹⁴ as an electrocatalyst POM attached to at least one biological carrier wherein
[X] is a diRuthenium sawhorse molecule represented by the formula (Ru₂(CO)₄(u-n²-O₂CR)₂)ⁿ wherein −3≦n≦6 and R represents hydrogen, hydroxide or a substituted or unsubstituted alkyl, heteroalkyl, aryl or heteroaryl group; and
L is a linking group that bonds the 2 [X] complexes together.

2. The composition of claim 1 wherein the biological carrier is selected from the group consisting of porphyrin, hemoglobin, heme-containing complexes, casein, and synthetically made carrier molecules.

3. The composition of claim 1 wherein L contains at least one of the elements selected from the group consisting of O, S, Se, and Te.

4. The composition of claim 1 wherein [X]-L-[X] is attached to at least one type of POM represented by the formula Na₁₄[Ru₂Zn₂(H₂O)₂(ZnW₉O₃₄)₂] and/or Na₁₄[Ru₂Zn₂(H₂O)₂(ZnW₉O₃₄)₂] then substituted to Ru₂Zn₂(H₂O)₂(ZnW₉O₃₄)₂⁻¹⁴ as an electrocatalyst POM, by an E-O bond wherein the E is selected from the group consisting of Si, Sn, and B.

5. The composition of claim 1 wherein at least one [X] of general formula [X]-L-[X] has at least one sulfonic acid or carboxylic acid group and said [X]-L-[X] is attached to at least one POM represented by the formula Na₁₄[Ru₂Zn₂(H₂O)₂(ZnW₉O₃₄)₂] and/or Na_{14,}[Ru₂Zn₂(H₂O)₂(ZnW₉O₃₄)₂] then substituted to Ru₂Zn₂(H₂O)₂(ZnW₉O₃₄)₂⁻¹⁴ as an electrocatalyst POM, by hydrogen bond interactions between said at least one sulfonic acid or carboxylic acid group of said [X]-L-[X] and a surface oxide ligand on said at least one POM.

6. The composition of claim 2 wherein said biological carrier is porphyrin and the composition has the formula 4Ru(III)Mesoporphyrin IX chloride (13-Bis(ethyl)-3,7,12,17-tetramethyl-21H,23H- porphine-2,18-dipropionic acid Tetraruthnate(III)chloride Mesohemin 8.

7. A composition for treating cancer comprising:
an effective amount for treating cancer of said composition of claim 1; and
one or more pharmaceutically acceptable carriers and/or adjuvants.

8. A composition for treating cancer comprising:
an effective amount for treating a cancerous growth of composition of claim 2; and
one or more pharmaceutically acceptable carriers and/or adjuvants.

9. A composition for treating cancer comprising:
an effective amount for treating a cancerous growth of composition of claim 3; and
one or more pharmaceutically acceptable carriers and/or adjuvants.

10. A composition for treating cancer comprising:
an effective amount for treating a cancerous growth of composition of claim 4; and
one or more pharmaceutically acceptable carriers and/or adjuvants.

11. A composition for treating cancer comprising:
an effective amount for treating a cancerous growth of composition of claim 5; and
one or more pharmaceutically acceptable carriers and/or adjuvants.

12. A composition for treating cancer comprising:
an effective amount for treating a cancerous growth of composition of claim 6; and
one or more pharmaceutically acceptable carriers and/or adjuvants.

13. A method for treating cancer in a mammal comprising administering to a mammal in need of cancer treatment a therapeutically effective amount of the composition of claim 7.

14. A method for treating cancer in a mammal comprising administering to a mammal in need of cancer treatment a therapeutically effective amount of the composition of claim 8.

14. A method for treating cancer in a mammal comprising administering to a mammal in need of cancer treatment a therapeutically effective amount of the composition of claim 9.

15. A method for treating cancer in a mammal comprising administering to a mammal in need of cancer treatment a therapeutically effective amount of the composition of claim 10.

16. A method for treating cancer in a mammal comprising administering to a mammal in need of cancer treatment a therapeutically effective amount of the composition of claim 11.

17. A method for treating cancer in a mammal comprising administering to a mammal in need of cancer treatment a therapeutically effective amount of the composition of claim 12.

18. The method of claim 13, wherein the mammal is a human.

19. The method of claim 19 wherein the cancer is selected from a group comprising rectal carcinoma, colon carcinoma, breast carcinoma, ovarian carcinoma, small cell lung carcinoma, colon carcinoma, chronic lymphocytic carcinoma, hairy cell leukemia, esophogeal carcinoma, prostate carcinoma, breast cancer, yeoman, and lymphoma.

20. The method of claim 19 wherein the cancer is a tumor of epithelial tissue, lymphoid tissue, connective tissue, bone, or central nervous system.

21. The method of claim 19 wherein the therapeutically effective amount is a daily dosage of about 0.90 to 1.1 mg /pound of Bodyweight for about 11 to about 16 days for two consecutive months with a dosage schedule of 4 days on said daily dosage and 3 days off said daily dosage.

22. The composition of claim 7 wherein the pharmaceutically acceptable carrier is a pharmaceutically acceptable propellant formulated for inhalable compositions.

23. The composition of claim 23 wherein said pharmaceutically acceptable propellant is selected from a group consisting of a gas, an aerosol, a solvent and mixtures thereof.

24. The composition of claim 24 wherein said solvent is selected from the group consisting of saline, polar solvents, non-polar solvents and mixtures thereof, wherein said composition further comprises between about 6 to about 34 Na+ ions bonded to said Ruthenium compound for use as a powder.

25. The method of claim 18 wherein the cancer being treated is selected from a group consisting of cancer of the ethmoidal plate and or tissue, cancer of Sphenoidal tissue, cancer of connective tissue, bone cancer, retroethmoidal cancer and retrospehnoidal cancers.

26. The method of claim 25 wherein an inhalable powder of said composition for treating cancer is bound to a bronchodilating drug and/or a steroidal compound for use in a mammal in need of cancer treatment.
